(19) United States
(12) Patent Application Publication
(10) Pub. No.: US 2003/0158227 A1
(43) Pub. Date: Aug. 21, 2003

(54) Polymorphs of fexofenadine base

(76) Inventors: Barnaba Krochmal, Jerusalem (IL); Dov Diller, Jerusalem (IL); Ben-Zion Dolitzky, Petach Tiqva (IL); Judith Aronhime, Rehovot (IL); Shlomit Wizel, Petah Tiqva (IL)

Correspondence Address: KENYON & KENYON, ONE BROADWAY, NEW YORK, NY, 10004, US

(21) Appl. No.: 10/290,655

(22) Filed: Nov. 8, 2002

Related U.S. Application Data
(60) Provisional application No. 60/336,930, filed on Nov. 8, 2001.
(60) Provisional application No. 60/339,041, filed on Dec. 7, 2001.
(60) Provisional application No. 60/344,114, filed on Dec. 28, 2001.
(60) Provisional application No. 60/361,780, filed on Mar. 4, 2002.
(60) Provisional application No. 60/363,482, filed on Mar. 11, 2002.
(60) Provisional application No. 60/387,670, filed on Jun. 10, 2002.
(60) Provisional application No. 60/390,198, filed on Jun. 19, 2002.
(60) Provisional application No. 60/403,765, filed on Aug. 15, 2002.
(60) Provisional application No. 60/406,214, filed on Aug. 27, 2002.

(51) Int. Cl. A61K 31/445; C07D 211/34

(52) U.S. Cl. 514/317; 546/238

(57) ABSTRACT

The present invention provides novel crystal forms of fexofenadine base and processes for their preparation. The forms are useful for administration to humans and animals to alleviate symptoms caused by histamine. The present invention further provides pharmaceutical compositions of the new crystalline forms.

### CROSS-REFERENCE TO RELATED APPLICATIONS

This application claims the benefit under 35 U.S.C. §119(e) of provisional applications Serial Nos. 60/336,930, filed Nov. 8, 2001; 60/339,041, filed Dec. 7, 2001; 60/344,114, filed Dec. 28, 2001; 60/361,780, filed Mar. 4, 2002; 60/363,482, filed Mar. 11, 2002; 60/387,670, filed Jun. 10, 2002; 60/390,198, filed Jun. 19, 2002, 60/403,765, filed Aug. 15, 2002; 60/406,214, filed Aug. 27, 2002 and claims the benefit under 35 U.S.C. §120 of U.S. patent application Ser. No. 10/133,460, filed Apr. 26, 2002, which is in turn a continuation-in-part of U.S. patent application Ser. No. 10/118,807, filed Apr. 8, 2002, all of which are incorporated herein by reference.

### FIELD OF THE INVENTION

The present invention relates to the solid state chemistry of fexofenadine base and its use as an active pharmaceutical agent.

### BACKGROUND OF THE INVENTION

4-[4-[4-(hydroxydiphenylmethyl)-1-piperidinyl]-1-hydroxybutyl]-α,α-dimethylbenzeneacetic acid of formula (I) (fexofenadine) is an H₁ receptor antagonist and a useful antihistaminic drug. It has low permeability into central nervous system tissues and weak antimuscarinic activity, causing it to have few systemic side effects.

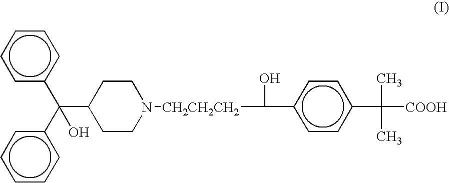

The antihistamic activity of fexofenadine was first disclosed in U.S. Pat. No. 4,254,129, incorporated herein by reference. According to the '129 patent, fexofenadine can be prepared starting from ethyl α,α-dimethylphenyl acetate and 4-chlorobutyroyl chloride, which are reacted under Freidel-Crafts conditions. Chloride is displaced from the Freidel-Crafts product with α,α-diphenyl-4-piperidinemethanol to give 4-[4-[4-(hydroxydiphenylmethyl)-1-piperidinyl]-1-oxobutyl]-α,α-dimethylbenzeneacetate, which is isolated as its hydrochloride salt. The ketone is then reduced with PtO/H₂ and the ester group is hydrolyzed to yield fexofenadine base.

Other methods of preparing fexofenadine are discussed in U.S. Pat. Nos. 5,578,610, 5,589,487, 5,581,011, 5,663,412, 5,750,703, 5,994,549, 5,618,940, 5,631,375, 5,644,061, 5,650,516, 5,652,370, 5,654,433, 5,663,353, 5,675,009, 5,375,693 and 6,147,216.

The present invention relates to the solid state physical properties of fexofenadine base prepared by any of these or other methods. These properties can be influenced by controlling the conditions under which fexofenadine base is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

The art discloses polymorphism of fexofenadine hydrochloride, rather than fexofenadine base. U.S. Pat. Nos. 5,738,872, 5,932,247 and 5,855,912, incorporated herein by reference, describe four crystal forms of fexofenadine hydrochloride which were designated Forms I-IV. According to the '872 and related patents, Forms II and IV are hydrates and Forms I and III are anhydrous. Each form was characterized by its melting point, onset of endotherm in the DSC profile, and PXRD. Form I is reported to have a capillary melting point range of 196-201° C., a DSC endotherm with onset between 195-199° C. and a powder X-ray diffraction (“PXRD”) pattern with d-spacings of 14.89, 11.85, 7.30, 6.28, 5.91, 5.55, 5.05, 4.96, 4.85, 4.57, 4.45, 3.94, 3.89, 3.84, 3.78, 3.72, 3.63, 3.07, 3.04, 2.45 Å. Form II is reported to have a capillary melting point range of 100-105° C., a DSC endotherm with onset between 124-126° C. and a PXRD pattern with d-spacings of 7.8, 6.4, 5.2, 4.9, 4.7, 4.4, 4.2, 4.1, 3.7, 3.6, 3.5 Å. Form III is reported to have a capillary melting point range of 166-171° C., a DSC endotherm with onset at 166° C. and a PXRD pattern with d-spacings of 8.95, 4.99, 4.88, 4.75, 4.57, 4.47, 4.46, 3.67, 3.65 Å. In Example 2, Form IV is reported to undergo decomposition at 115-116° C. In the general written description, a DSC endotherm with onset at 146° C. is reported. Form IV is reported as having a PXRD pattern with d-spacings of 10.38, 6.97, 6.41, 5.55, 5.32, 5.23, 5.11, 4.98, 4.64, 4.32, 4.28, 4.12, 4.02, 3.83, 3.65, 3.51, 3.46 and 2.83 Å.

The '872 patent discusses methods of interconverting Forms I-IV. Aqueous recrystallization of Form I can be used to produce Form II. Water-minimizing recrystallization or azeotropic distillation of either Form II or Form IV can yield Form I. Form III is reported to be accessible by water minimizing recrystallization of Form II. Crystal digestion of Form III can be used to obtain Form I. Forms II and IV can be obtained directly by sodium borohydride reduction of 4-[4-[4-(hydroxydiphenylmethyl)-1-piperidinyl]-1-oxobutyl]-α,α-dimethylbenzeneacetate as described in Examples 1 and 2.

International Publication No. WO 00/71124 A1, discloses that amorphous fexofenadine hydrochloride can be prepared by lyophilizing or spray drying a solution of fexofenadine hydrochloride. The product is characterized by its IR spectrum and a featureless PXRD pattern.

International Publication No. WO 01/94313 is also directed to polymorphs of fexofenadine hydrochloride.

The present invention provides new crystal forms of fexofenadine base, rather than fexofenadine hydrochloride, and processes for their preparation.

### SUMMARY OF THE INVENTION

In one aspect, the present invention provides for a new crystalline form of fexofenadine base, which is characterized by a powder X-ray diffraction pattern with peaks at about 9.8, 11.6, 12.1, 13.5, 14.0, 18.0, 18.4 and 19.7±0.2 degrees two theta, and/or a differential scanning calorimetric thermogram with a major endothermic peak at about 100° C. and a minor endothermic peak at about 143° C., and two exothermic peaks at about 155° C. and about 180° C. Said new crystalline form denotes Form I.

In another aspect, the present invention provides a process for preparing fexofenadine base having at least one of the characteristics of Form I comprising the steps of preparing a solution of fexofenadine base in 1-propanol, admixing the solution with water, ice or a mixture thereof to form a precipitate and separating the precipitate as fexofenadine base Form I.

In another aspect, the present invention provides for fexofenadine base which is characterized by a PXRD pattern with peaks at about 7.4, 9.7, 11.7, 12.1, 13.8, 14.4, 18.0, 18.5 and 19.7±0.2 degrees two theta and/or a differential scanning calorimetric thermogram with an endotherm at about 100° C., a maximum endotherm at about 223° C. and a minor endotherm at about 144° C., and two exotherms at about 146° C. and about 182° C. Said new crystalline form denotes Form II.

In another aspect, the present invention provides a process for preparing fexofenadine base having at least one of characteristics of Form II comprising the steps of preparing a solution of fexofenadine base in a mixture of water and 1-propanol, wherein fexofenadine base precipitates from the solution, and separating the fexofenadine base.

In another aspect, the present invention provides for fexofenadine base which is characterized by a PXRD pattern with peaks at about 4.4, 10.3, 11.3, 16.3, 19.8±0.2 degrees 2θ and a DSC thermogram with two exothermic peaks at about 107° C. and about 166° C., and an endotherm at about 226° C. Said new crystalline form denotes Form III.

In another aspect, the present invention provides a process for preparing fexofenadine base having at least one of characteristics of Form III comprising the steps of slurrying fexofenadine base in methanol, heating the slurry and separating fexofenadine base Form III as a solid.

In another aspect, the present invention provides for foxofenadine base characterized by a PXRD diffraction pattern with peaks at about 4.3, 8.7, 12.5, 13.1 and 13.6±0.2 degrees 2θ, which is further characterized by peaks at about 16.3, 16.7, 17.5, 18.1, 18.5, 19.6, 20.7, 21.8 and 22.6±0.2 degrees 2θ. Said new crystalline form denotes Form IV.

In another aspect, the present invention provides a process for preparing fexofenadine base having one of characteristics of Form IV comprising the steps of preparing a solution of fexofenadine base in a mixture of a C₁ to a C₄ alcohol and water, with the proviso that the alcohol is not 1-propanol, wherein fexofenadine base precipitates from the solution and separating the precipitate; A process for preparing fexofenadine base having one of characteristics of Form IV comprising the steps of preparing a solution of a sodium or a potassium salt of fexofenadine in a mixture of a C₁ to a C₄ alcohol and water, with the proviso that the alcohol is not 1-propanol and acidifying the solution to precipitate fexofenadine base. Preferably methanol is used.

In another aspect, the present invention provides for fexofenadine base which is characterized by a PXRD pattern with peaks at about 17.2, 18.2, 18.8, 20.3±0.2 deg. 2θ and/or a DSC thermogram with an endotherm followed by an exotherm at about 200° C., and an additional endotherm at about 226° C. Said new crystalline form denotes Form V.

In another aspect the present invention provides a process for preparing fexofenadine base having at least one of characteristics of Form V comprising the steps of slurrying fexofenadine base in methyl ethyl ketone and separating fexofenadine base Form V as a solid.

In another aspect, the present invention provides for fexofenadine base which is characterized by a PXRD pattern (FIG. 9) with peaks at about 3.9, 9.6, 11.8, 16.0 and 19.0±0.2 degrees 2θ, and/or a DSC thermogram (FIG. 10) with endotherms at about 140° C. and 229° C., and an exotherm at about 160° C. Said new crystalline form denotes Form VI.

In another aspect the present invention provides processes for preparing fexofenadine base having at least one of characteristics of Form VI comprising the steps of slurrying fexofenadine base in methanol under suitable condition, separating a solid from the methanol and optionally repeating the slurry process.

In another aspect, the present invention provides for fexofenadine base, which is characterized by a PXRD pattern (FIG. 11) with peaks at about 3.9, 7.7, 10.6, 13.4, 14.5 and 19.2±0.2 degrees 2θ and/or a DSC thermogram (FIG. 12) with an endotherm at about 228° C. Said new crystalline form denotes Form VII.

In another aspect, the present invention provides processes for preparing fexofenadine base having at least one of characteristics of Form VII comprising carrying out an azeotropic distillation of fexofenadine base in toluene to remove water.

In another aspect, the present invention provides for pharmaceutical compositions of fexofenadine base and their methods of administration.

### BRIEF DESCRIPTION OF THE FIGURES

FIG. 1 is a PXRD pattern for fexofenadine Base Form I.

FIG. 2 is a DSC thermogram for fexofenadine base Form I.

FIG. 3 is a PXRD pattern for fexofenadine base Form II.

FIG. 4 is a DSC thermogram for fexofenadine base Form II.

FIG. 5 is a PXRD pattern for fexofenadine base Form III.

FIG. 6 is a DSC thermogram for fexofenadine base Form III.

FIG. 7 is a PXRD pattern for fexofenadine base Form V.

FIG. 8 is a DSC thermogram for fexofenadine base Form V.

FIG. 9 is a PXRD pattern for fexofenadine base Form VI.

FIG. 10 is a DSC thermogram for fexofenadine base Form VI.

FIG. 11 is a PXRD pattern for fexofenadine base Form VII.

FIG. 12 is a DSC thermogram for fexofenadine base Form VII.

### DETAILED DESCRIPTION OF THE INVENTION

As used herein, “about A to B” refers to “about A to about B” unless otherwise specified.

As used herein, precipitation (or “precipitate”) is used in the same way as crystallization (or “crystal”), and refers to obtaining a solid material from a solution.

As used herein, “slurry” is used the same way as “suspension”.

As used herein, the term “Fexofenadine base Form X” refers to a polymorph of fexofenadine base that one of skill in the art can identify as a distinct entity distinguishable from other polymorphs of fexofenadine base based on the characterization provided herein. The term “having at least one of characteristics of Form X” refers to a crystalline fexofenadine base polymorph that possesses one of the PXRD peaks, or endotherms and exotherms of a DSC thermogram provided herein. For example, a single or a combination of PXRD peaks which is not found in another polymorphic entity of fexofenadine base is enough to show at least one of the characteristics of Form X. A single or a combination of endotherms and/or exotherms of a DSC thermogram may also serve the same purpose.

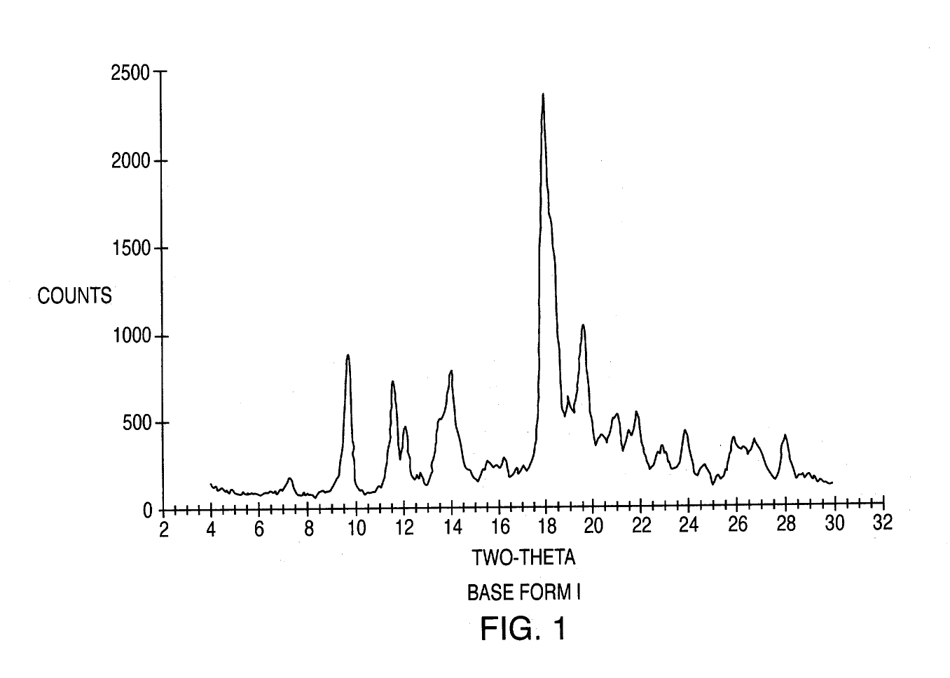

In one aspect, the present invention provides new crystal Form I of fexofenadine base (designated Form X in Provisional Appl. No. 60/336930, filed Nov. 8, 2001, entitled “Crystal Forms X, XI, XII and XIII of Fexofenadine Hydrochloride, Compositions Containing the New Forms and Methods of Relieving Inflammation by Administering the New Forms”). The fexofenadine base Form I has a PXRD pattern (FIG. 1) with characteristic peaks at about 9.8, 11.6, 12.1, 13.5, 14.0, 18.0, 18.4 and 19.7±0.2 degrees two theta.

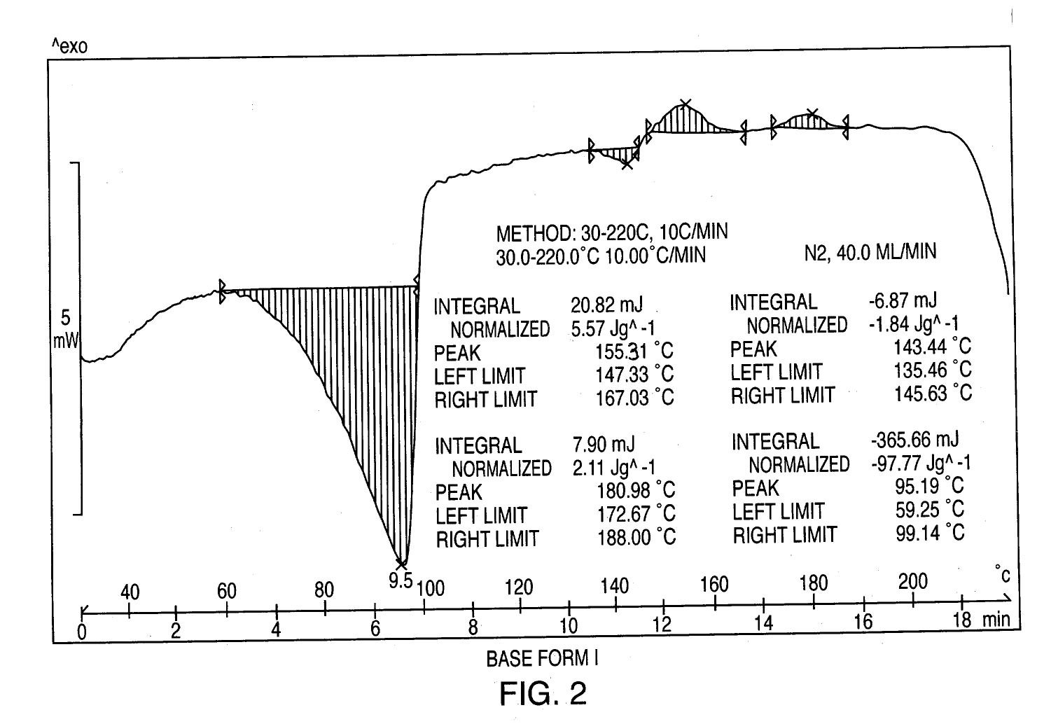

Fexofenadine base Form I has a DSC profile (FIG. 2) characterized by a major endothermic peak at about 100° C. and a minor endothermic peak at about 143° C., and two exothermic peaks at about 155° C. and about 180° C.

In another aspect, the present invention provides for a process for preparing fexofenadine base having at least one of characteristics of Form I comprising the steps of preparing a solution of fexofenadine base in 1-propanol, admixing the solution with water or ice or a mixture thereof to form a precipitate and separating the precipitate.

Fexofenadine base is added to 1-propanol. The temperature may be raised to further dissolve the fexofenadine base in 1-propanol. Preferably, the temperature is raised to more than about 60° C., more preferably about 80° C. One of skill in the art would also appreciate that other temperatures may be adequate to dissolve the fexofenadine base in 1-propanol and that other temperatures may be used under other conditions.

After the fexofenadine base has been dissolved, the solution is poured into ice, water or a mixture thereof to cause precipitation of fexofenadine base Form I. Preferably, the water is kept at about room temperature.

The precipitated fexofenadine base Form I is separated from the solution. One of skill in the art would appreciate that there are many ways to separate the precipitate from the solution. Preferably, the precipitate is separated with a filter. The separated precipitate may be optionally dried under either ambient or reduced pressure. In a preferred embodiment, the precipitate is dried under a vacuum.

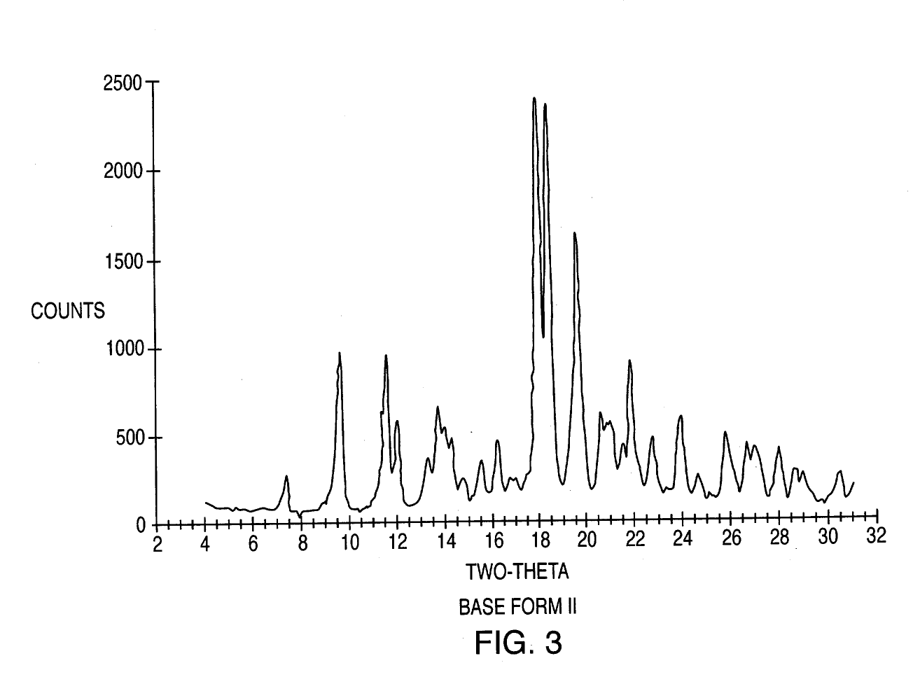

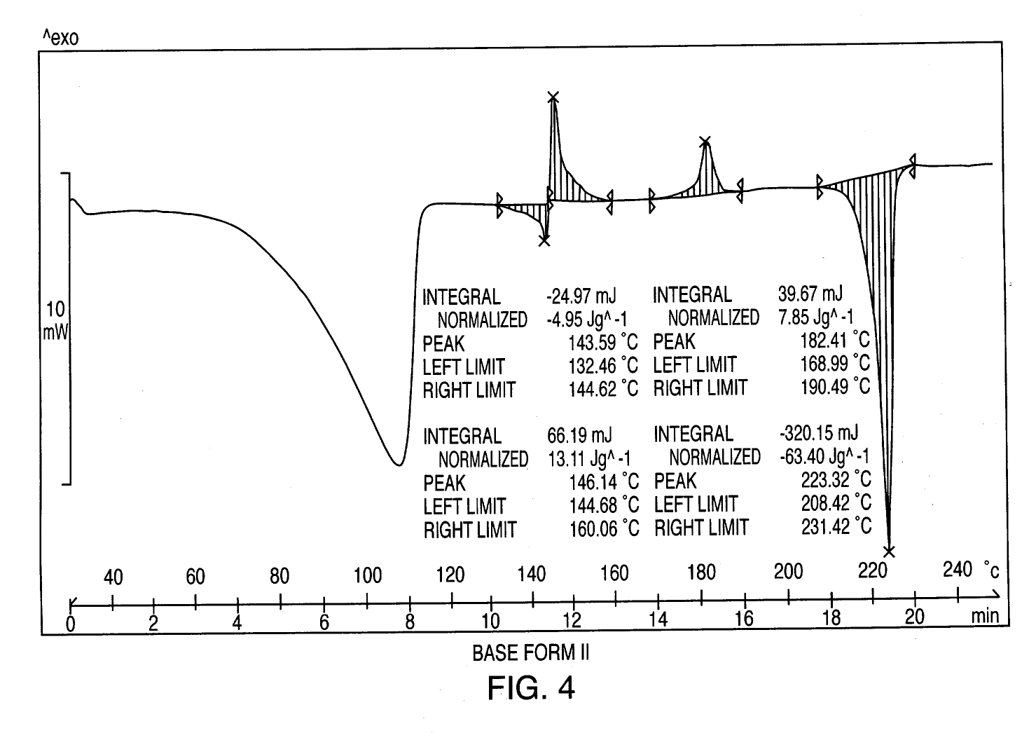

The present invention also provides fexofenadine base Form II (designated Form XI in Provisional Appl. No. 60/336,930, filed Nov. 8, 2001, entitled “Crystal Forms X, XI, XII and XIII of Fexofenadine Hydrochloride, Compositions Containing the New Forms and Methods of Relieving Inflammation by Administering the New Forms”). The fexofenadine base Form II has a powder X-ray diffraction pattern (FIG. 3) with peaks at about 7.4, 9.7, 11.7, 12.1, 13.8, 14.4, 18.0, 18.5 and 19.7±0.2 degrees two theta. The fexofenadine base Form II also has a differential scanning calorimetric (DSC) thermogram (FIG. 4) having an endotherm at about 100° C., a maximum endotherm at about 223° C. and a minor endotherm at about 144° C., and two exotherms at about 146° C. and about 182° C.

Fexofenadine base Form II may be prepared by preparing a solution of fexofenadine base in a mixture of water and 1-propanol, wherein fexofenadine base precipitates from the solution and separating the fexofenadine base.

First, a solution of fexofenadine base is prepared in a mixture of 1-propanol and water. The mixture of water and 1-propanol is preferably from about a 4:1 ratio to about a 1:1 ratio (vol/vol). Most preferably, the mixture is about a 3:1 ratio of water to 1-propanol. Preferably, the mixture is heated to completely dissolve the fexofenadine base. Preferably, the temperature is raised to more than about 60° C., more preferably to about 80° C.

After dissolution, the solution is preferably cooled to about room temperature and left for a few days to crystallize. Water is used for preparation of Form II as a co-solvent of the solution, rather than as an anti-solvent added to the solution as in preparation of Form I. After crystallization, the crystals are separated, preferably by a filter, and optionally dried. To accelerate the drying process, the temperature may be raised or the pressure reduced. Preferably, the crystals are dried from about 55° C. to about 70° C. in a vacuum. A vacuum oven known in the art may be used. The product of this process is fexofenadine free base II.

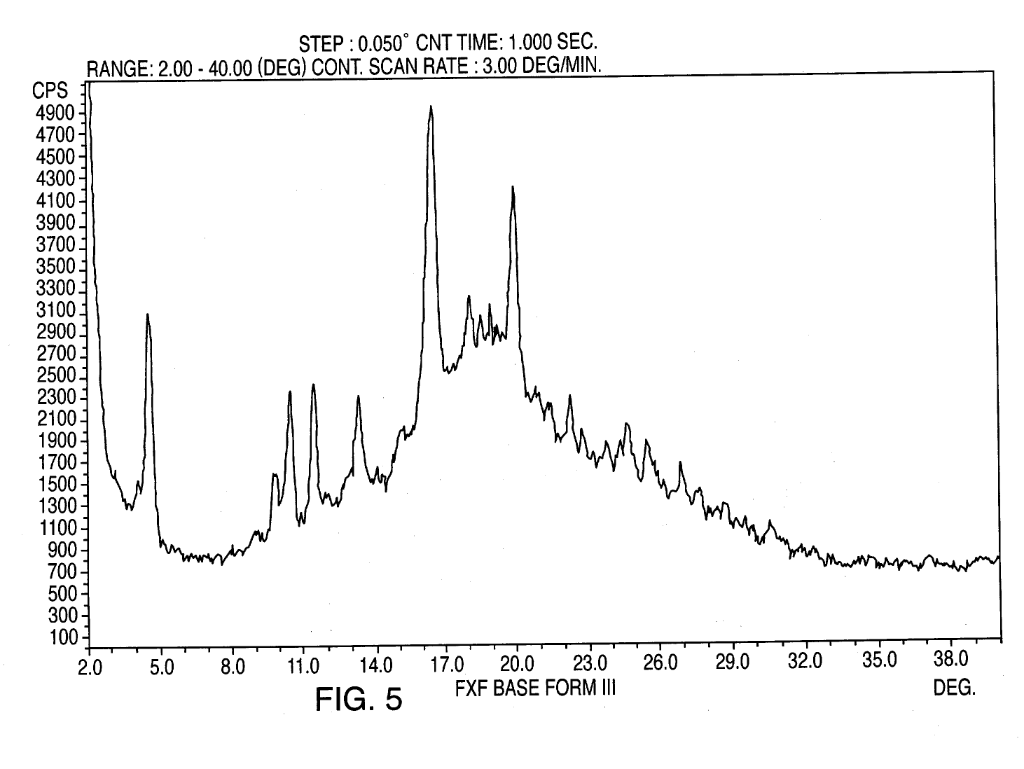

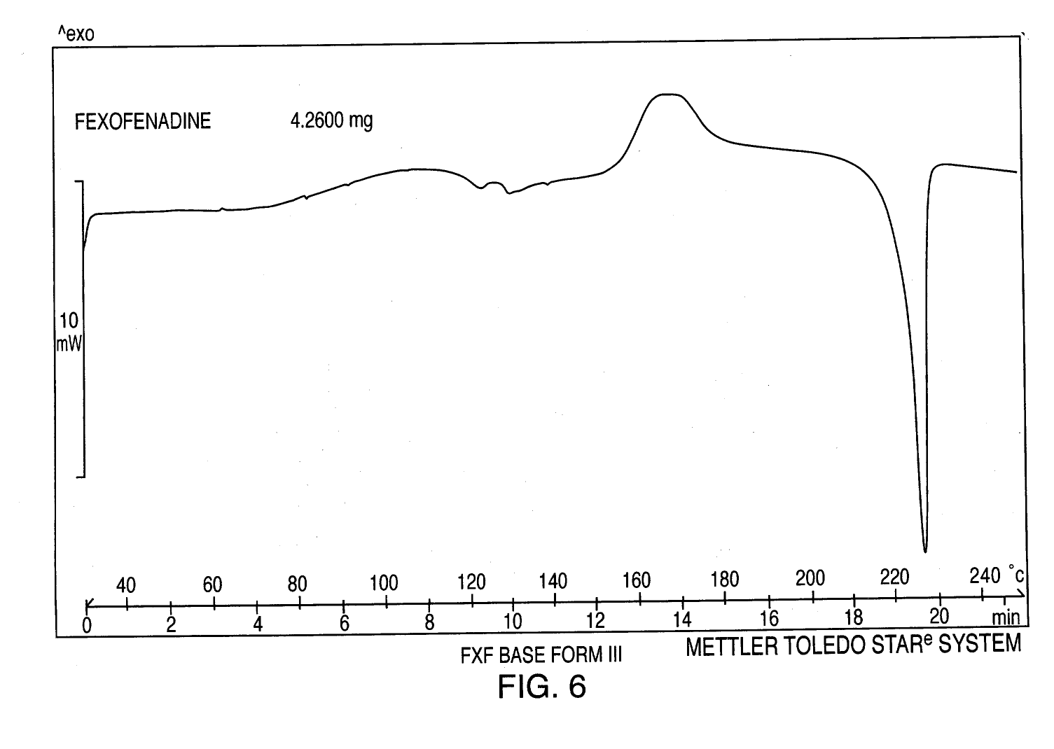

In another aspect, the present invention provides for fexofenadine base Form III. Fexofenadine base Form III is characterized by a PXRD pattern (FIG. 5) with peaks at about 4.4, 10.3, 11.3, 16.3, 19.8±0.2 degrees 2θ. Fexofenadine free base Form III is also characterized by a DSC thermogram (FIG. 6) with two broad exothermic peaks at about 107° C. and about 166° C., and a sharp endotherm at about 226° C.

In another aspect the present invention provides a process for preparing fexofenadine base having at least one of characteristics of Form III comprising the steps of slurrying fexofenadine base in methanol, heating the slurry and separating fexofenadine base Form III as a solid.

Fexofenadine free base is slurried in methanol and heated for a sufficient amount of time at a suitable temperature to obtain a transition to Form III. Preferably, the slurry is heated from about 5 minutes to about 50 hours at about reflux (65° C.), more preferably for less than about 1 hour. Preferably, the temperature is above about 45° C., with reflux temperature being most preferred. The slurry is then preferably cooled. Fexofenadine base Form III settles down, which is then separated by techniques well known in the art, such as filtration. The fexofenadine base is optionally dried. The temperature can be increased or the pressure reduced to accelerate the drying process. Preferably, the fexofenadine base is dried at about 60° C. overnight. The product of this process is fexofenadine base Form III.

The present invention also provides for fexofenadine base Form IV. Fexofenadine base Form IV is characterized by a PXRD pattern with peaks at about 4.3, 8.7, 12.5, 13.1, 13.6, 16.3, 16.7, 17.5, 18.1, 18.5, 19.6, 20.7, 21.8 and 22.6±0.2 degrees 2θ. The most characteristic peaks are at about 4.3, 8.7, 12.5, 13.1 and 13.6±0.2 degrees 2θ.

Fexofenadine base Form IV can be prepared by a process comprising the steps of preparing a solution of fexofenadine base in a mixture of a C₁ to a C₄ alcohol and water, with the proviso that the alcohol is not 1-propanol, wherein fexofenadine base precipitates from the solution and separating the precipitate.

First a solution of fexofenadine base is prepared in a mixture of water and a lower alcohol, preferably methanol. Fexofenadine base Form IV is then crystallized out of the solution. The solution can be cooled for example to allow for precipitation. To prepare the solution of fexofenadine base, a first solution of the sodium or potassium salt of fexofenadine base can be prepared, by using a suitable inorganic base such as potassium or sodium hydroxide. After obtaining a solution of the salt of fexofenadine, the solution is acidified to precipitate fexofenadine base. One of skill in the art would appreciate that such precipitation theoretically goes through a solution of fexofenadine base, even if only momentarily. Acids known in the art such as acetic acid can be used to acidify the first solution. Before acidification, the pH of the solution is preferably above about 8. The pH of the solution after acidification is preferably maintained above about 3.5, more preferably from about 4 to about 7. The precipitate can then be separated by techniques well known in the art such as filtration.

The resulting precipitate, fexofenadine free base Form IV, can then optionally be slurried, and the slurry process can be repeated as desired. Suitable solvents for slurry include lower alcohols such as methanol. The optional slurry process is preferably for a limited time, less than about 2 hours, to avoid a transition of Form IV to other forms; Form IV is relatively stable during slurry for short amount of times. Form IV can also be recrystallized from a mixture of an alcohol and a ketone, such as a methanol:methyl ethyl ketone (“MEK”) mixture. Preferably, the slurry is carried out at a temperature of from about 45° C. to about 65° C., more preferably at about 50° C. Residual solvents can be removed by drying the precipitate.

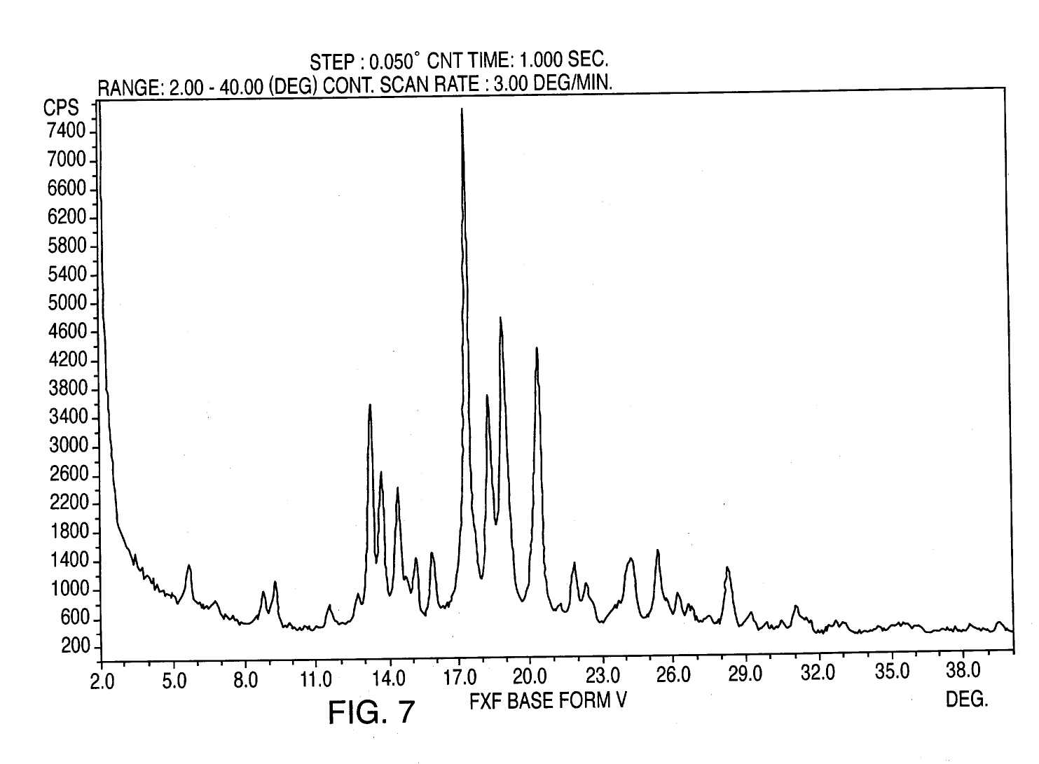

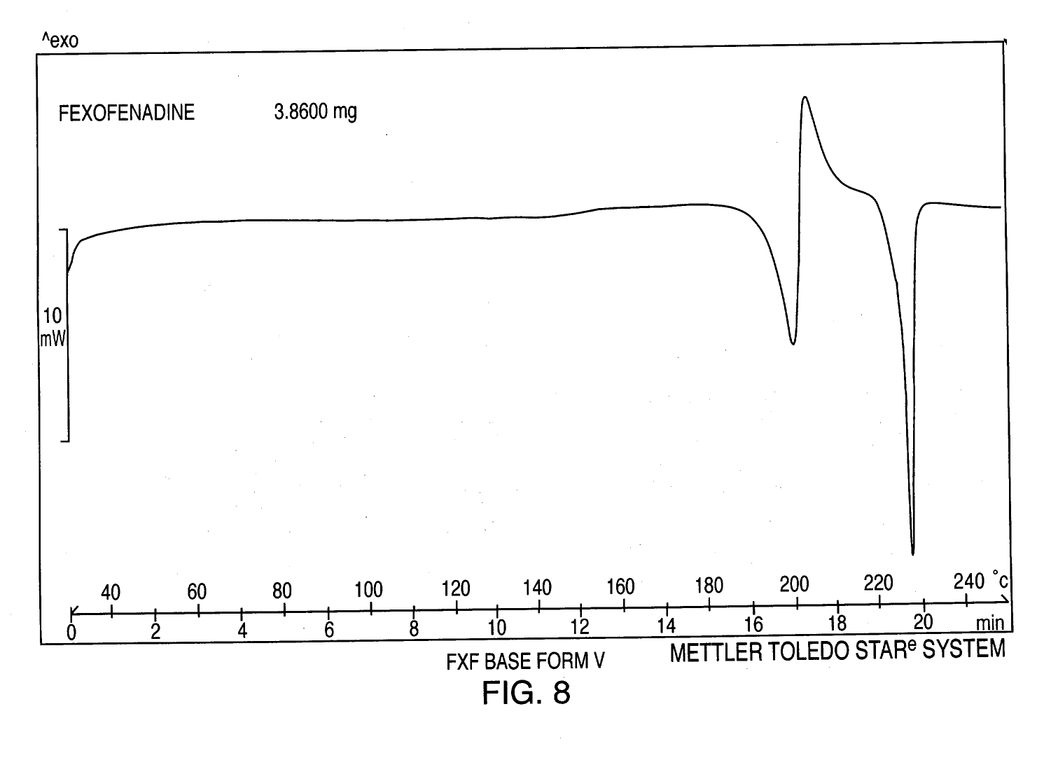

In another aspect, the present invention provides for fexofenadine base Form V. Fexofenadine base Form V is characterized by a PXRD diffraction pattern (FIG. 7) with peaks at about 13.2, 13.7, 14.4, 17.2, 18.2, 18.8, 20.3±0.2 degrees 2θ. The most characteristic peaks are at about 17.2, 18.2, 18.8, 20.3±0.2 degrees 2θ. Fexofenadine base is also characterized by a DSC thermogram (FIG. 8) with an endotherm followed by an exotherm at about 200° C., and an additional endotherm at about 226° C.

In another aspect, the present invention provides a process for preparing fexofenadine base having at least one of characteristics of Form V comprising the steps of slurrying fexofenadine base in methyl ethyl ketone and separating fexofenadine base Form V as a solid.

Fexofenadine free base is slurried in methyl ethyl ketone and preferably heated to reflux. The slurry is then preferably cooled. Fexofenadine base Form V is then separated as a solid, preferably by filtration. The separated foxofenadine base is then optionally dried. The pressure is reduced or the temperature increased to accelerate the drying process. Preferably, fexofenadine base is dried at a temperature of about 65° C. in a vacuum. The product of this process is fexofenadine base Form V.

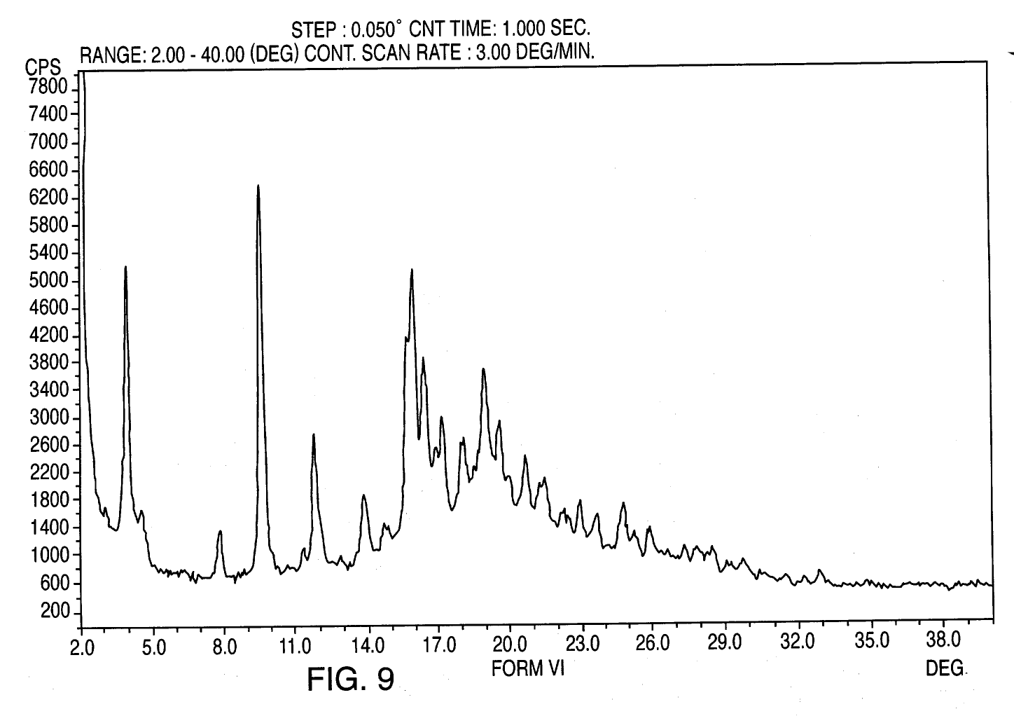

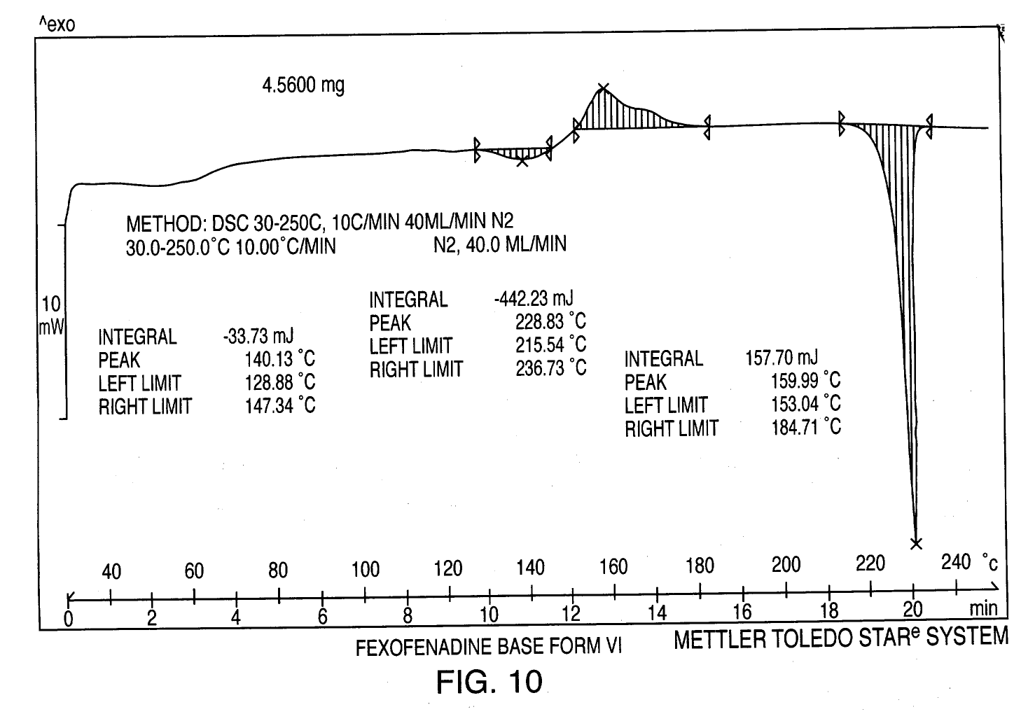

The present invention provides for a new form of fexofenadine base, labeled Form VI. Fexofenadine Form VI is characterized by a PXRD pattern (FIG. 9) with peaks at about 3.9, 9.6, 11.8, 16.0 and 19.0±0.2 degrees 2θ. The DSC profile of fexofenadine Form VI (FIG. 10) has an endotherm at about 140° C. followed by an exotherm. In addition, the DSC profile has a sharp endothermic peak at about 229° C.

Fexofenadine base Form VI can be prepared by slurry of fexofenadine base in methanol. Fexofenadine base is added to methanol and slurried for preferably a few hours. A solid is then separated from the slurry by conventional techniques such as filtration. In one embodiment, the slurry process is repeated at least once, more preferably twice, by adding the separated solid to methanol and slurrying the solid. After slurrying, a solid is separated for a final time. Before separation, the solid is preferably allowed to stand for a while. The separated solid is then optionally dried, from about 40° C. to about 70° C., more preferably about 60° C.

The slurry of fexofenadine base in methanol in the process for preparation of From VI is carried out under suitable conditions, i.e., preferably not heated above room temperature to avoid a transition to Form III. One of skill in the art would appreciate that the transition depends on the temperature and the amount of time of heating, and that these variable can be ascertained in a routine fashion. Thus, Form VI may also be obtained with slight heating, as long as the heating is not sufficient to induce a transition to Form III. In the preferred embodiment, heating is not applied at all, while in another the temperature is kept below about 30° C. The starting material used for preparing either Form III or Form VI is preferably not fexofenadine Form IV since it tends to show relative stability towards the slurry process when slurried for short periods of times.

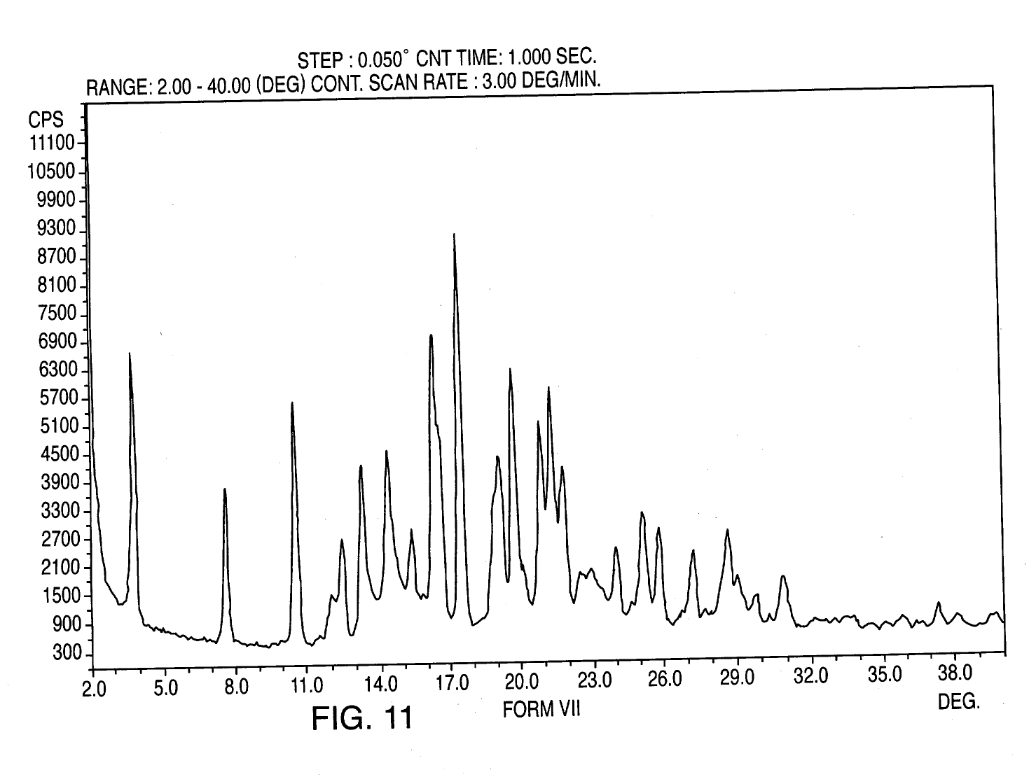

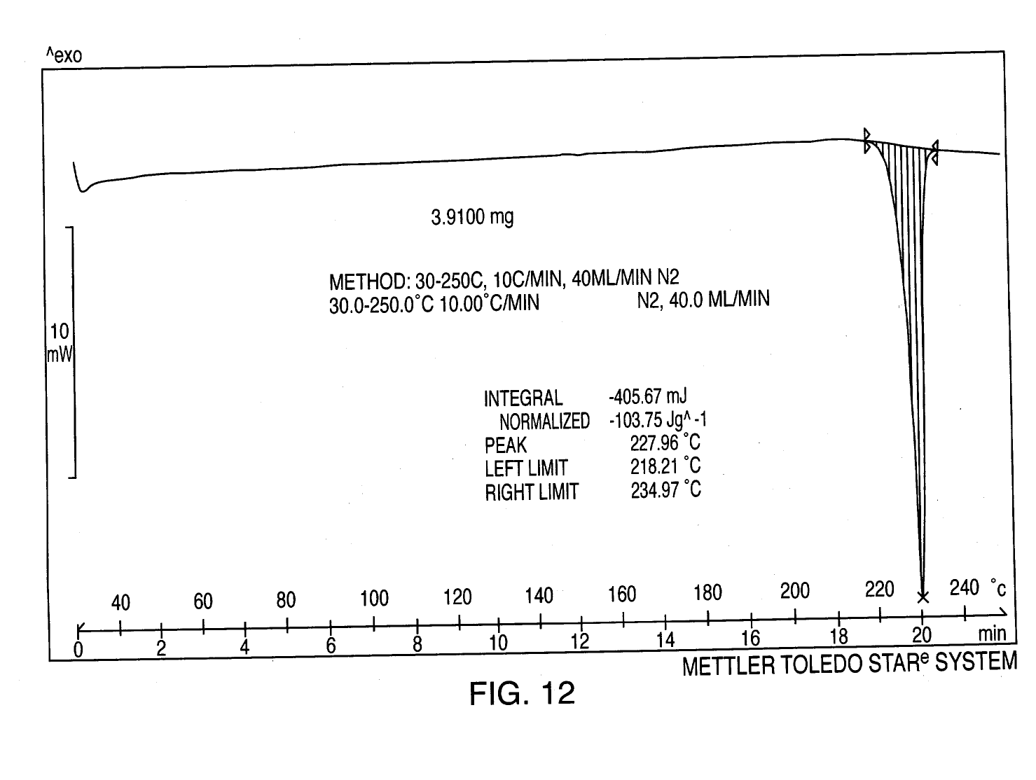

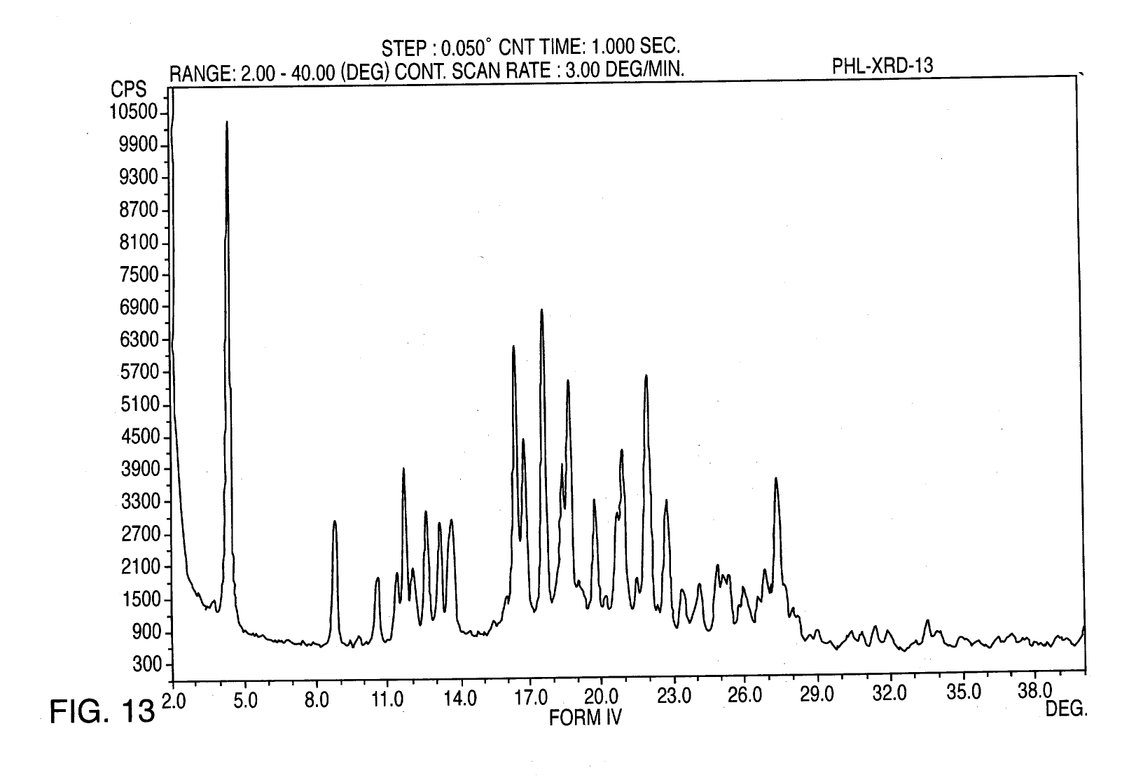

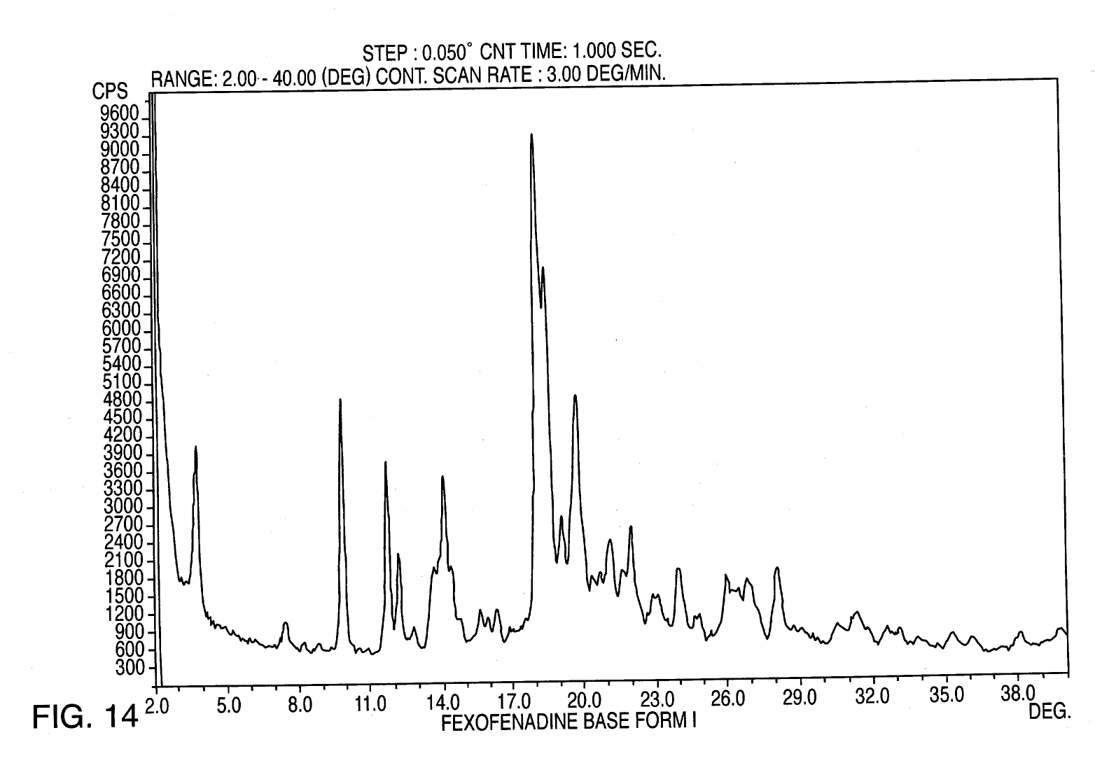

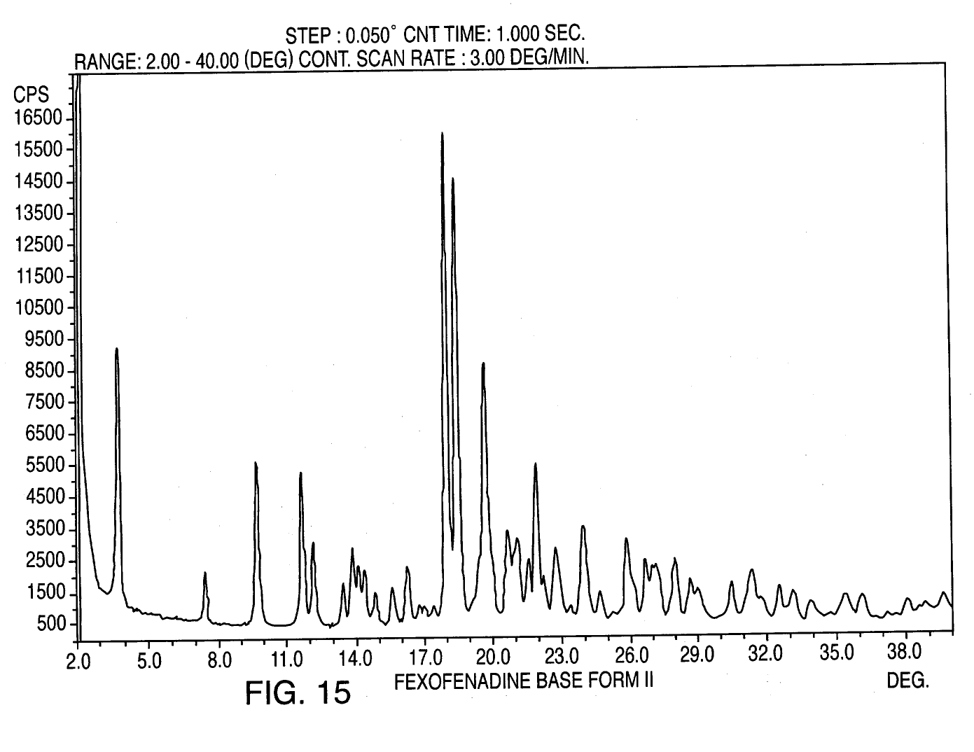

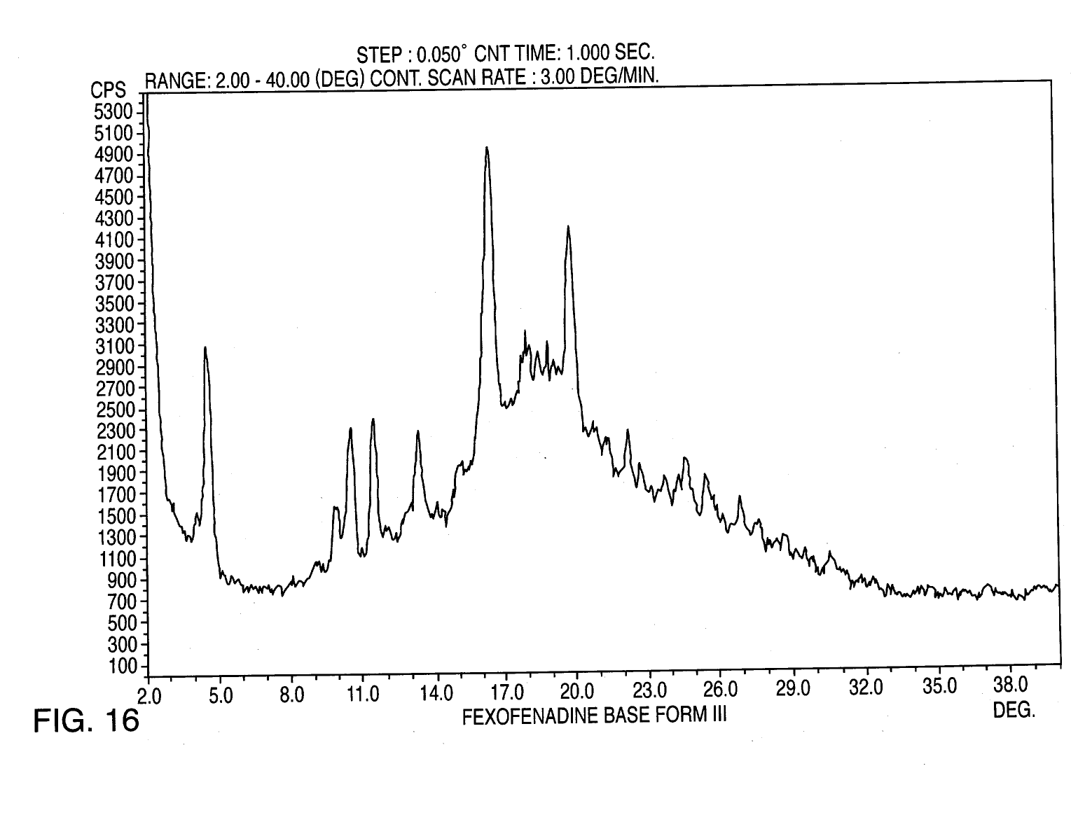

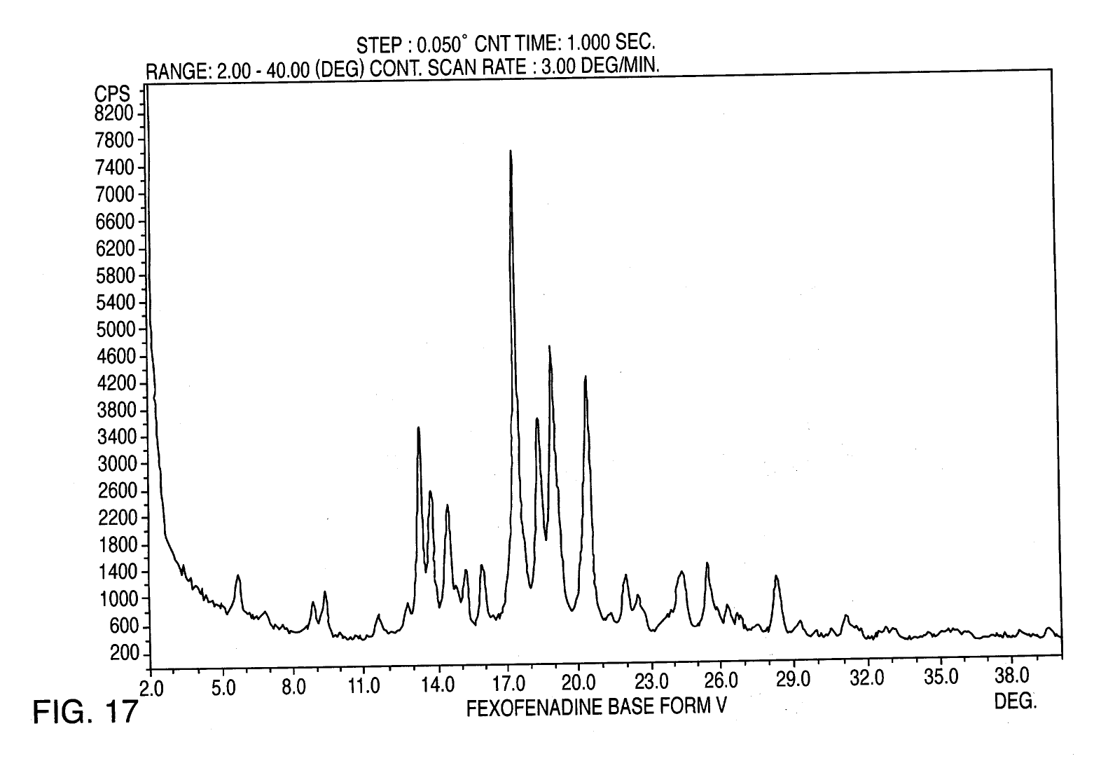

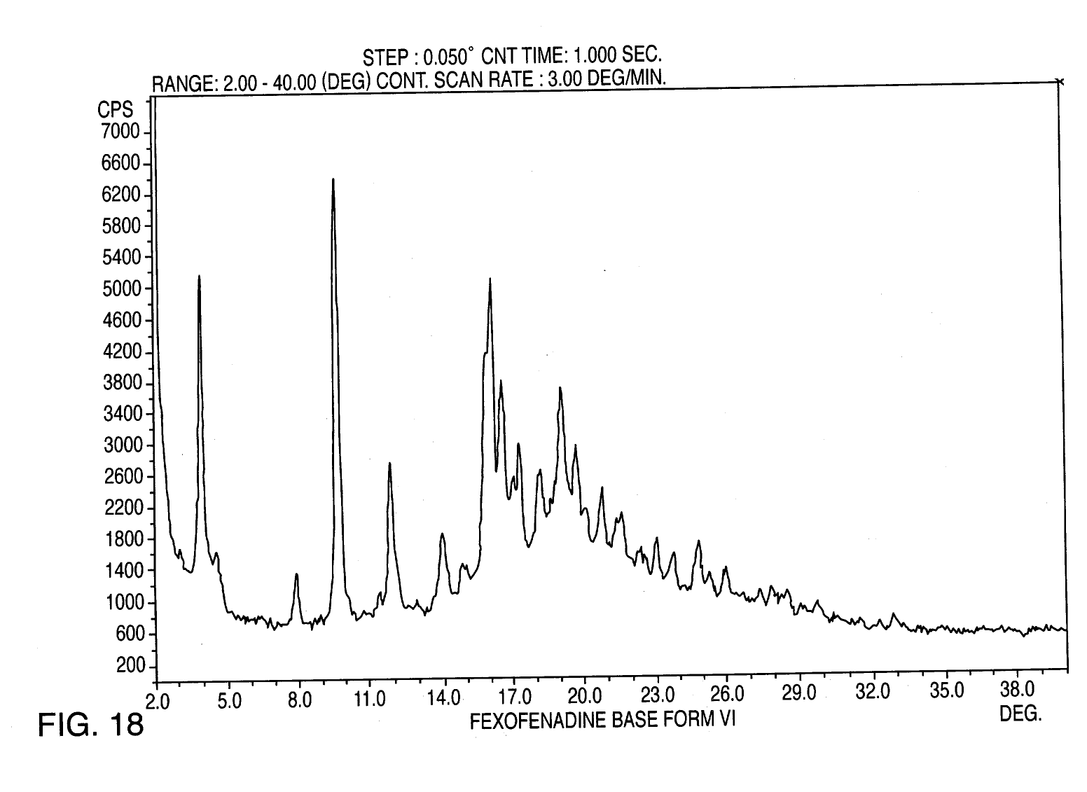

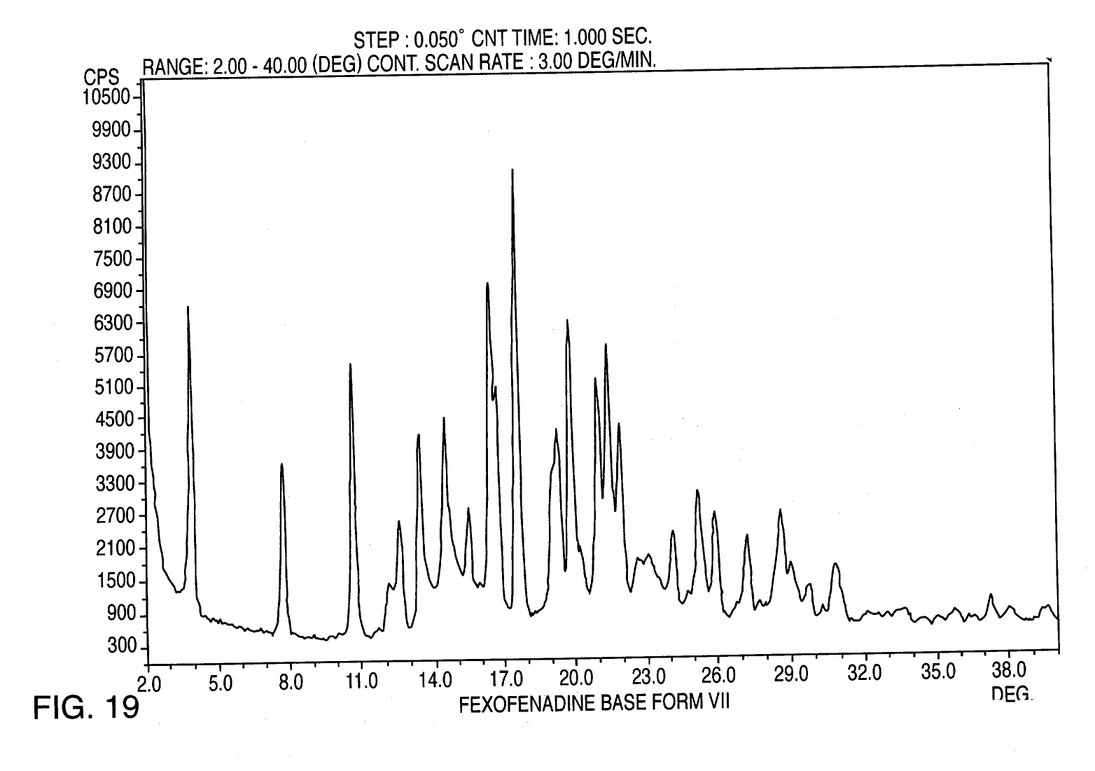

The present invention also provides for fexofenadine base Form VII. Fexofenadine base Form VII is characterized by a PXRD pattern (FIG. 11) with peaks at about 3.9, 7.7, 10.6, 13.4, 14.5 and 19.2±0.2 degrees 2θ. Fexofenadine base Form VII is also characterized by a DSC thermogram (FIG. 12) with an endotherm at about 228° C.

The present invention also provides a process for preparing fexofenadine base having at least one of characteristics of Form VII comprising carrying out an azeotropic distillation of fexofenadine in toluene to remove water. The water removed is traces of water present in the starting material. A hydrate can also be used as a starting material, where the water of crystallization is removed as well. To carry out the process, fexofenadine base is added of toluene. The mixture is then distilled to remove the water. After removal of the water, the mixture is preferably allowed to cool. The fexofenadine can then be separated by conventional techniques, preferably filtration. The wet sample can then optionally be dried, preferably from a temperature of about 40° C. to about 70° C., with about 60° C. being preferred. Before drying, the solid can be recrystallized from a suitable solvent such as a lower alcohol.

One of skill in the art would appreciate that the polymorphs of the present invention can be selectively obtained generally through crystallization with different recrystallization solvent systems. The starting material can be anhydrous fexofenadine base or any fexofenadine base hydrate or lower alcohol solvate. The starting fexofenadine base can also be in an amorphous or any crystalline crystal form. The starting material can also be that prepared by reducing a fexofenadine ketoacid with a catalytic reducing reagent or a transfer hydride such as sodium borohydride, BH₃ or lithium borohydride. The process can be used as a purification method by using the desired form in an unacceptably pure state as starting material.

The various crystal forms of fexofenadine free base of the present can be converted to the hydrochloride salt. The examples and the art provide proper guidance for such conversion. Hydrochloric acid used can be aqueous or non-aqueous. The aqueous hydrochloric acid used is preferably concentrated and has a molarity of about 12 or a mass percentage of about 38%. Preferably, hydrochloric acid is used in a slight excess, more particularly from about a 1.01 to about a 1.20 molar equivalent of the free base. The free base can be regenerated by treating the salt with a suitable dilute aqueous base solution, such as dilute aqueous sodium hydroxide, potassium carbonate, ammonia or sodium bicarbonate.

Many processes of the present invention involve crystallization out of a particular solvent. One of skill in the art would appreciate that the conditions concerning crystallization can be modified without affecting the form of the polymorph obtained. For example, when mixing free base in a solvent to form a solution, warming of the mixture can be necessary to completely dissolve the starting material. If warming does not clarify the mixture, the mixture can be diluted or filtered. To filter, the hot mixture can be passed through paper, glass fiber or other membrane material, or a clarifying agent such as celite. Depending upon the equipment used and the concentration and temperature of the solution, the filtration apparatus may need to be preheated to avoid premature crystallization.

The conditions can also be changed to induce precipitation. A preferred way of inducing precipitation is to reduce the solubility of the solvent. The solubility of the solvent can be reduced, for example, by cooling the solvent.

In one embodiment, an anti-solvent is added to a solution to decrease its solubility for a particular compound, thus resulting in precipitation. In another embodiment, an anti-solvent is added to an oily residue or a gummy material, wherein the low solubility of the anti-solvent for a particular compound results in precipitation of that compound.

Another manner to accelerate crystallization is by seeding with a crystal of the product or scratching the inner surface of the crystallization vessel with a glass rod. Other times, crystallization can occur spontaneously without any inducement. The present invention covers both embodiments where precipitation is induced/accelerated or occurs spontaneously, except in the circumstance where the inducement/acceleration is critical for obtaining a particular polymorph, e.g., the process requires the use of a particular anti-solvent. A separate precipitating step is not recited in the present invention to emphasize that precipitation can occur spontaneously, but such emphasis is not meant to change the scope of the present invention from one reciting a separate precipitating step.

As an antihistamine, fexofenadine is effective at relieving symptoms caused by airborne and contact inducers of histamine release. Such substances include pollen, spores, animal dander, cockroach dander, industrial chemicals, dust and dust mites. Symptoms that can be alleviated by fexofenadine include bronchial spasms, sneezing, rhinorrhia, nasal congestion, lacrimation, redness, rash, urticaria and itch.

Fexofenadine base Forms I, II, III, IV, V, VI and VII are useful for delivering fexofenadine to the gastrointestinal tract, mucus membranes, bloodstream and inflamed tissues of a patient suffering from inflammation caused by a histamine. They can be formulated into a variety of compositions for administration to humans and animals.

Pharmaceutical compositions of the present invention contain fexofenadine base Forms I, II, III, IV, V, VI and VII, optionally in a mixture with other forms or amorphous fexofenadine and/or active ingredients such as pseudoephedrine. They can also be optionally mixed with pseudoephedrine. In addition to the active ingredient(s), the pharmaceutical compositions of the present invention can contain one or more excipients. Excipients are added to the composition for a variety of purposes.

Diluents increase the bulk of a solid pharmaceutical composition and can make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form like a tablet can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of non-compacted solid composition and improve the accuracy of dosing. Excipients that can function as glidants include colloidal silicon dixoide, magnesium trisilicate, powdered cellulose, starch, talc and tribasic calcium phosphate.

When a dosage form such as a tablet is made by compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product form the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, fexofenadine base Forms I, II, III, IV, V, VI and VII, and any other solid excipients are dissolved or suspended in a liquid carrier such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste.

Preservatives and chelating agents such as alcohol, sodium benzoate, butylated hydroxy toluene, butylated hydroxyanisole and ethylenediamine tetraacetic acid can be added at levels safe for ingestion to improve storage stability.

A liquid composition according to the present invention can also contain a buffer such as guconic acid, lactic acid, citric acid or acetic acid, sodium guconate, sodium lactate, sodium citrate or sodium acetate.

Selection of excipients and the amounts to use can be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages can be conveniently presented in unit dosage form and prepared by any of the methods well-known in the pharmaceutical arts.

Dosage forms include solid dosage forms like tablets, powders, capsules, suppositories, sachets, troches and losenges as well as liquid syrups, suspensions and elixirs.

A dosage form of the present invention is a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

Capsules, tablets and lozenges and other unit dosage forms preferably contain a dosage level of about 30 to about 180 mg of fexofenadine base. Other dosages may also be administered depending on the need.

The following describes the instrumentation used by the present invention to characterize the new polymorphs. PXRD patterns were obtained by methods known in the art using a Scintag X-ray powder diffractometer, a variable goniometer, an X-Ray tube with Cu target anode (Cu radiation λ=1.5418 Å) and a solid state detector. A round standard aluminum sample holder with a round zero background quartz plate was used. Scans were performed over a range of 2 to 40 degrees two-theta, continuously, with a scan rate of 3 degrees/min.

Some of the samples, except for Forms III, IV, V, VI or VII were either obtained by using the above instrument or Philips XRD, a Goniometer Model 1050/70, a copper tube and a curved graphite monochromate. Same scanning parameters were used in such case.

The DSC thermogram was obtained using a DSC Mettler 821 Star. The temperature range of scans was 30-350° C. at a rate of 10° C./min. The weight of the sample was 2-5 mg. The sample was purged with nitrogen gas at a flow rate of 40 mL/min. Standard 40 μl aluminum crucibles having lids with three small holes were used.

The following examples are provided for illustration:

### EXAMPLE 1

Preparation of Fexofenadine Base Form I

Fexofenadine base (10 grams) was added to 1-propanol (50 mL). The solution was then heated to 80° C. in a hot water bath. After heating, the hot solution was poured onto ice (400 grams) with stirring. The solution was stirred for two days at 25° C. to form a precipitate. The precipitate was filtered and dried at 62° C. to yield 7.8 grams of product. Subsequent PXRD analysis confirmed that the product was a new form of fexofenadine base, labeled Form I.

### EXAMPLE 2

Preparation of Fexofenadine Base Form I

Fexofenadine base (10 grams) was dissolved at 80° C. in 1-propanol (150 mL). The resulting solution was then added slowly to deionized water (350 mL) kept at room temperature to precipitate crystals. The crystals were then filtered and dried under vacuum at a temperature less than 45° C. to obtain product. Subsequent PXRD analysis confirmed that the product was a new form of fexofenadine base, labeled Form I.

### EXAMPLE 3

Preparation of Fexofenadine Base Form II

Fexofenadine base (10 grams) was mixed with 1-propanol (30 mL) and water (100 mL). The mixture was then heated to 80° C. 1-propanol (40 mL) was then added to the mixture to complete the dissolution. The solution was cooled to 25° C. with stirring for 2 days to precipitate crystals. The crystals were filtered and dried under vacuum at 62° C. to yield 6.5 grams of product. Subsequent PXRD analysis confirmed that the product was a new form of fexofenadine base, labeled Form II.

### EXAMPLE 4

Preparation of Fexofenadine Base Form III

Fexofenadine base (80.2 grams) was slurried in methanol (400 mL) and heated at reflux for 20 minutes. The hot slurry was cooled to a temperature of 20° C. A solid was then filtered after 0.5 hours and dried overnight at 60° C. The yield of this process was 92%. Subsequent PXRD analysis confirmed the product was a new form of fexofenadine base, labeled Form III.

### EXAMPLE 5

Preparation of Fexofenadine Base Form IV

Fexofenadine ketoacid was reduced with sodium borohydride in methanol-water in the presence of sodium hydroxide. After completion of the reduction, the fexofenadine so prepared was precipitated by acidification using acetic acid. The resulting precipitate was filtered and slurried three times in methanol at 50° C. The material was dried for 2 hours at 65° C. under vacuum.

### EXAMPLE 6

Preparation of Fexofenadine Base Form IV

Fexofenadine free base was prepared an in Example 8 except that it was slurried only once in methanol and recrystallized from MEK:methanol. Fexofenadine free base (5 grams) was heated in boiling MEK:methanol 1:1 (300 ml), most of which dissolved. The mixture was filtered hot from undissolved material. The filtrate was left overnight at room temperature. A crystalline precipitate formed, which was cooled in an ice salt bath and filtered. The precipitate fexofenadine free base was dried for 1 hour under vacuum at 65° C.

Note: The material before crystallization was 98.9% pure by HPLC. The material which did not dissolve in MEK-methanol, was fexofenadine, which did not dissolve because of an insufficient amount of solvent.

### EXAMPLE 7

Preparation of Fexofenadine Base Form V

Fexofenadine free base (4.5 grams) was slurried in methyl ethyl ketone (70 mL) at reflux temperature for 2 hours. The hot slurry was then cooled to a temperature of 20° C. A solid settled down which was then filtered after 0.5 hours and dried in a vacuum oven at 65° C., first with a water aspirator and then with an oil vacuum pump. The yield of this process was 45%. Subsequent PXRD analysis confirmed the product was a new form of fexofenadine base, labeled Form V.

### EXAMPLE 8

Preparation of Fexofenadine Base Form VI

Fexofenadine base (50.3 grams) was slurried with 300 ml methanol at room temperature (˜25° C.) for 2.5 hours, followed by filtration. The filtered sample was slurried for a second time for 1.5 hours at room temperature with methanol, followed by filtration. The sample was slurried for a third time for 1.67 hours at room temperature with 300 ml of methanol. The sample was left alone without stirring for 12 hours, then filtered, and dried at 60° C. overnight to yield 9.5 grams of fexofenadine free base From VI. Subsequent PXRD analysis confirmed the product of this process was a new form of fexofenadine free base, labeled Form VI.

### EXAMPLE 9

Preparation of Fexofenadine Base Form VII

Fexofenadine base (30.52 grams) was set up in a dean stark apparatus using toluene (200 ml) to remove water. After 6.5 hours, the apparatus was allowed to cool. After standing overnight, a gray solid was filtered (3.46 grams), which was shown to be form VII as a wet sample. The wet sample was initially highly soluble in methanol, but crystallized out of methanol overtime. The wet sample (4.28 grams) was dried in the oven at 60° C. degrees for 3 hours to yield 2.22 grams of fexofenadine base Form VII. Subsequent PXRD analysis confirmed the product of this process was a new form of fexofenadine free base, labeled Form VII.

What is claimed is:

1. Fexofenadine base Form I.

2. A fexofenadine base in a crystalline form characterized by a PXRD pattern with peaks at about 9.8, 11.6, 12.1, 13.5, 14.0, 18.0, 18.4 and 19.7±0.2 degrees two theta.

3. The fexofenadine base of claim 2 having a PXRD pattern substantially as depicted in FIG. 1.

4. A fexofenadine base in a crystalline form having a differential scanning calorimetric thermogram with an endothermic peak at about 100° C. and an endothermic peak at about 143° C., and two exothermic peaks at about 155° C. and about 180° C.

5. A process for preparing fexofenadine base having at least one of characteristics of Form I comprising the steps of:
a) preparing a solution of fexofenadine base in 1-propanol;
b) admixing the solution with water, ice or a mixture thereof to precipitate fexofenadine base; and
c) separating the precipitate.

6. The fexofenadine base prepared by the process of claim 5.

7. Fexofenadine base Form II.

8. A fexofenadine base in a crystalline form having a PXRD pattern with peaks at about 7.4, 9.7, 11.7, 12.1, 13.8, 14.4, 18.0, 18.5 and 19.7±0.2 degrees two theta.

9. The fexofenadine base of claim 8 having a powder PXRD pattern substantially as depicted in FIG. 3.

10. A fexofenadine base in a crystalline form having a differential scanning calorimetric thermogram with an endotherm at about 100° C., a maximum endotherm at about 223° C. and a minor endotherm at about 144° C., and two exotherms at about 146° C. and about 182° C.

11. A process for preparing fexofenadine base having at least one of characteristics of Form II comprising the steps of:
a) preparing a solution of fexofenadine base in a mixture of water and 1-propanol, wherein fexofenadine base precipitates from the solution; and
b) separating the fexofenadine base.

12. The process of claim 11, wherein the mixture is from about a 1:1 to about a 4:1 mixture (vol/vol) of water and 1-propanol.

13. The process of claim 12, wherein the mixture is about a 3:1 mixture of water and 1-propanol.

14. The fexofenadine base prepared by the process of claim 11.

15. Fexofenadine base Form III.

16. A fexofenadine base in a crystalline form characterized by a PXRD diffraction pattern with peaks at about 4.4, 10.3, 11.3, 16.3, 19.8±0.2 degrees 2θ.

17. The fexofenadine base of claim 16 having a PXRD pattern as substantially depicted in FIG. 5.

18. A fexofenadine base in a crystalline form characterized by a DSC thermogram with an endotherm followed by an exotherm at about 200° C., and an additional endotherm at about 226° C.

19. The fexofenadine base of claim 18 characterized by a DSC thermogram with two exothermic peaks at about 107° C. and about 166° C., and an endotherm at about 226° C.

20. A process for preparing fexofenadine base having at least one of characteristics of Form III comprising the steps of:
a) slurrying fexofenadine base in methanol;
b) heating the slurry; and
c) separating fexofenadine base Form III as a solid.

21. The fexofenadine base prepared by the process of claim 20

22. The process of claim 20, wherein heating involves a temperature of from about 45° C. to about reflux.

23. The process of claim 20, wherein the fexofenadine base used in step (a) is not fexofenadine base Form IV.

24. Fexofenadine base Form IV.

25. Fexofenadine base in a crystalline form characterized by a PXRD diffraction pattern with peaks at about 4.3, 8.7, 12.5, 13.1 and 13.6±0.2 degrees 2θ.

26. The fexofenadine base of claim 25, further characterized by peaks at about 16.3, 16.7, 17.5, 18.1, 18.5, 19.6, 20.7, 21.8 and 22.6±0.2 degrees 2θ.

27. A process for preparing fexofenadine base having one of characteristics of Form IV comprising the steps of:
a) preparing a solution of fexofenadine base in a mixture of a C₁ to a C₄ alcohol and water, with the proviso that the alcohol is not 1-propanol, wherein fexofenadine base precipitates from the solution; and
b) separating the precipitate.

28. The process of claim 27, wherein the alcohol is methanol.

29. The process of claim 27 or 28, wherein preparing a solution involves preparing a first solution of a sodium or potassium salt of fexofenadine followed by acidification of the first solution.

30. A process for preparing fexofenadine base having one of characteristics of Form IV comprising the steps of:
a) preparing a solution of a sodium or a potassium salt of fexofenadine in a mixture of a C₁ to a C₄ alcohol and water, with the proviso that the alcohol is not 1-propanol; and
b) acidifying the solution to precipitate fexofenadine base.

31. The process of claim 30, wherein the alcohol is methanol.

32. The process of claim 30 or 31, wherein the solution has a pH of more than about 8.

33. The process of claim 30 or 31, wherein acidifying results in a pH of from about 4 to about 7.

34. The fexofenadine base prepared by the process of claim 27 or 30.

35. Fexofenadine base Form V.

36. A fexofenadine base in a crystalline form characterized by a PXRD diffraction pattern with peaks at about 17.2, 18.2, 18.8, 20.3±0.2 degrees 2θ.

37. The fexofenadine base of claim 36 further characterized by a PXRD pattern with peaks at about 13.2, 13.7, 14.4±0.2 degrees 2θ.

38. The fexofenadine base of claim 37 further characterized by a PXRD pattern as substantially depicted in FIG. 7.

39. A fexofenadine base in a crystalline form characterized by a DSC thermogram with an endotherm followed by an exotherm at about 200° C., and an additional endotherm at about 226° C.

40. A process for preparing fexofenadine base having at least one of characteristics of Form V comprising the steps of:
a) slurrying fexofenadine base in methyl ethyl ketone; and
b) separating fexofenadine base Form V as a solid.

41. The fexofenadine base prepared by the process of claim 40.

42. Fexofenadine base Form VI.

43. A fexofenadine base in a crystalline form characterized by a PXRD pattern with peaks at about 3.9, 9.6, 11.8, 16.0 and 19.0±0.2 degrees 2θ.

44. The fexofenadine base of claim 43 further characterized by a PXRD pattern as substantially depicted in FIG. 9.

45. A fexofenadine base in a crystalline form characterized by a DSC thermogram with endotherms at about 140° C. and about 229° C., and an exotherm at about 160° C.

46. A process for preparing fexofenadine base having at least one of characteristics of Form VI comprising the steps of:
a) slurrying fexofenadine base in methanol under suitable condition; and
b) separating fexofenadine base Form VI as a solid.

47. The process of claim 46, further comprising repeating steps (a) and (b) at least once.

48. The process of claim 46, wherein step (a) is carried out at a temperature of about 30° C. or below.

49. The process of claim 46, wherein the fexofenadine base used in step (a) is not fexofenadine base Form IV.

50. A Fexofenadine base Form VII.

51. A fexofenadine base in a crystalline form characterized by a PXRD pattern with peaks at about 3.9, 7.7, 10.6, 13.4, 14.5 and 19.2±0.2 degrees 2θ.

52. The fexofenadine base of claim 50 further characterized by a PXRD pattern as substantially depicted in FIG. 11.

53. A fexofenadine base in a crystalline form characterized by a DSC thermogram with an endotherm at about 228° C.

54. A process for preparing fexofenadine base having at least one of characteristics of Form VII comprising carrying out an azeotropic distillation of fexofenadine base in toluene to remove water.

55. The process of claim 54, further comprising recrystallizing the fexofenadine base from methanol.

56. A pharmaceutical composition comprising an effective amount of fexofenadine selected from the group consisting of base Forms I, II, III, IV, V, VI and VII, and a pharmaceutically acceptable excipient.

57. A method of inhibiting binding between an H₁ receptor and histamine in a patient suffering from contraction of the bronchi, vasodilation, itching or other inflammation response to histamine comprising administering to the patient the pharmaceutical composition of claim 56.

58. A method of alleviating symptoms of allergic rhinitis in a patient susceptible to allergic rhinitis or experiencing symptoms of allergic rhinitis comprising administering to the patient the pharmaceutical composition of claim 56.
